(19) United States
(12) Patent Application Publication
(10) Pub. No.: US 2006/0293367 A1
(43) Pub. Date: Dec. 28, 2006

(54) Amorphous N-(2-((4-hydroxyphenyl)amino)pyridin-3-yl)-4-methoxybenzenesulfonamide

(76) Inventor: Geoff G.Z. Zhang, Libertyville, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/332,275

(22) Filed: Jan. 13, 2006

Related U.S. Application Data
(60) Provisional application No. 60/643,559, filed on Jan. 13, 2005.

(51) Int. Cl. C07D 211/72; A61K 31/44

(52) U.S. Cl. 514/352; 546/304

(57) ABSTRACT

An amorphous drug, processes for making it, compositions containing it and methods of treatment of diseases using it are disclosed.

This application claims priority to co-pending U.S. Provisional Application Ser. No. 60/643,559, filed Jan. 13, 2005.

### FIELD OF THE INVENTION

This invention pertains to amorphous N-(2-((4-hydroxyphenyl)amino)pyridin-3-yl)-4-methoxybenzenesulfonamide, processes for making it, compositions containing it and methods of treatment of diseases and inhibition of adverse physiological events using it.

### BACKGROUND OF THE INVENTION

Crystallinity of drugs, or lack thereof, impacts both their manufacture and their utility. There is therefore an existing need in the chemical and therapeutic arts for an understanding of physical properties of crystalline and amorphous forms of drugs and ways to reproducibly make them.

### SUMMARY OF THE INVENTION

One embodiment of this invention pertains to amorphous N-(2-((4-hydroxyphenyl)amino)pyridin-3-yl)-4-methoxybenzenesulfonamide.

Another embodiment pertains to substantially amorphous N-(2-((4-hydroxyphenyl)amino)pyridin-3-yl)-4-methoxybenzenesulfonamide.

Still another embodiment pertains to amorphous N-(2-((4-hydroxyphenyl)amino)pyridin-3-yl)-4-methoxybenzenesulfonamide having substantial chemical purity.

Still another embodiment a composition comprising an excipient and a therapeutically acceptable amount of amorphous N-(2-((4-hydroxyphenyl)amino)pyridin-3-yl)-4-methoxybenzenesulfonamide.

Still another embodiment pertains to a method for treating cancer in a mammal comprising administering thereto a therapeutically effective amount of amorphous N-(2-((4-hydroxyphenyl)amino)pyridin-3-yl)-4-methoxybenzenesulfonamide.

Still another embodiment pertains to a process for making substantially amorphous N-(2-((4-hydroxyphenyl)amino)pyridin-3-yl)-4-methoxybenzenesulfonamide, said process comprising milling N-(2-((4-hydroxyphenyl)amino)pyridin-3-yl)-4-methoxybenzenesulfonamide, with or without solvent, until said substantially amorphous N-(2-((4-hydroxyphenyl)amino)pyridin-3-yl)-4-methoxybenzenesulfonamide develops.

Still another embodiment pertains to substantially amorphous N-(2-((4-hydroxyphenyl)amino)pyridin-3-yl)-4-methoxybenzenesulfonamide made by the process of the preceeding embodiment.

### DETAILED DESCRIPTION OF THE INVENTION

This invention pertains to amorphous N-(2-((4-hydroxyphenyl)amino)pyridin-3-yl)-4-methoxybenzenesulfonamide, ways to identify it, ways to make it having substantial chemical and geometric purity, compositions containing it and methods of treatment of diseases using it.

The term “amorphous,” as used herein, means a supercooled liquid or a viscous liquid wthat looks like a solid but does not have a regularly repeating arrangement of molecules that is maintained over a long range and does not have a melting point but rather softens or flows above its glass transition temperature.

The term “substantially amorphous,” as used herein, means at least about 95% amorphous, preferably about 98% amorphous, and more preferably about 100% amorphous.

The term “milling,” as used herein, means grinding between two surfaces.

Milling can be conducted with a mortar and pestle or a milling process such as ball milling, roller milling, or gravatory milling.

The term “substantial chemical purity,” as used herein, means about 95% chemical purity, preferably about 97% chemical purity, more preferably about 98% chemical purity, and most preferably about 100% chemical purity.

The term “chemical purity,” as used herein, means percentage of a particular compound in a sample. A sample of amorphous N-(2-((4-hydroxyphenyl)amino)pyridin-3-yl)-4-methoxybenzenesulfonamide may contain, for example, acetic acid, ethanol, ethyl acetate, isopropyl acetate, isopropyl ether, methanol, n-propanol, pyridine, pyridine hydrochloride, water, 4-aminophenol, 3,4-bis(4-hydroxyanilino)-6-((4-hydroxyphenyl)imino)-2,4-cyclohexadien-1-one of varying geometric purity, 2-chloro-3-nitropyridine or a regioisomer thereof, 2,6-di-tert-butylphenol, 4-((3-nitro-2-pyridinyl)oxy)aniline, para-methoxybenzenesulfonyl chloride, 4-((3-(((4-methoxyphenyl)sulfonyl)amino)pyridin-2-yl)amino)phenyl 4-methoxybenzenesulfonate or a mixture thereof.

Unless stated otherwise, percentages throughout this specification are weight/weight (w/w) percentages.

The term “solvent,” as used herein, means a substance, preferably a liquid or a miscible, partially miscible or immiscible mixture of two or more liquids, which is capable of partially dissolving, suspending, dispersing or partially dispersing another substance, preferably a solid or a mixture of solids.

It is meant to be understood that, because many solvents and anti-solvents contain impurities, the level of impurities in solvents and anti-solvents for the practice of this invention, if present, are at a low enough concentration that they do not interfere with the intended use of the solvent in which they are present.

Amorphous N-(2-((4-hydroxyphenyl)amino)pyridin-3-yl)-4-methoxybenzenesulfonamide may be made as follows.

### EXAMPLE 1

A mixture of 2-chloro-3-nitropyridine (2C3NP, 138.1 Kg), 4-aminophenol (2.5-3 equivalents) and N,N-dimethylformamide (DMF, 4.8 mL/g 2C3NP) was stirred until homogeneous, heated at 50° C. during which an exotherm raised the solution temperature to 70° C., warmed to 80-85° C., stirred until no 2-chloro-3-nitropyridine remained, cooled to 30° C., treated with water (10.6 mL/g 2C3NP) to precipitate product, then with acetic acid (1.2 mL/g 2C3NP), then with ethyl acetate (0.5 mL/g 2C3NP), cooled to 5° C., stirred for 2 hours and filtered. The filtrant was washed sequentially with distilled water (1.6 mL/g 2C3NP), cold ethanol (1.2 mL/g 2C3NP) and cold isopropyl ether (1.2 mL/g 2C3NP), and dried under vacuum.

In a preferred embodiment of this process, 4-aminophenol (1 equivalent) was used with 4-methylmorpholine (1.5 equivalents) in either methanol or DMF, and precipitation was accomplished with 10% aqueous acetic acid.

### EXAMPLE 2

A mixture of EXAMPLE 1 (41.05 Kg) and ammonium formate (5 equivalents), with or without 2,6-di-tert-butylphenol antioxidant, was treated with a mixture of 50% wet 5% palladium hydroxide on carbon (7% by weight per weight of EXAMPLE 1), in DMF (6 mL/g catalyst) then DMF (total DMF volume: 5 mL/g EXAMPLE 1) first with moderate agitation to control an exotherm (typically peaking at 85° C.) then with increased agitation for 1 hour (incomplete reactions were treated with additional catalyst/DMF mixture), cooled to 10° C., and filtered. The filtrant was washed with DMF (0.4 mL/g EXAMPLE 1), and the filtrate was added to water (29.4 mL/g EXAMPLE 1) at 10° C. to precipitate a solid which was filtered, washed with water (7.5 mL/g EXAMPLE 1), partially dried under a nitrogen stream, and further dried under vacuum at 50° C. to about 0.5% moisture.

### EXAMPLE 3

A mixture of EXAMPLE 2 in pyridine (9 mL/g) at 0° C. was treated with a mixture of para-methoxybenzenesulfonyl chloride (1.05 equivalents) in THF (1.4 mL/g) at 0° C. at a rate which kept the reaction temperature below 5° C., warmed to 25° C., stirred for 15 minutes, and concentrated. The concentrate was treated with n-propanol to provide a composition having 9% pyridine in the solvent mixture and to precipitate a solid. The mixture was cooled to 0° C. and filtered. The filtrant and washed with ethyl acetate (5-7 mL/g starting material) and dried at 45° C.

### EXAMPLE 4

A mixture of EXAMPLE 3 and saturated aqueous sodium bicarbonate (2 equivalents) was extracted with ethyl acetate (6 mL/g EXAMPLE 3). The extract was washed with brine (4 mL/g EXAMPLE 3), treated with n-propanol (2 mL/g EXAMPLE 4), and concentrated until the ethyl acetate was present in less than 1%. The concentrate was adjusted to 70:30 n-propanol:water (150-180 mg EXAMPLE 4/g solution), and the hot solution was filtered through a 0.2 micrometer filter. The filtrate was adjusted to solvent composition of 60:40 n-propanol:water and a concentration of approximately 130 mg product/g solution, cooled slowly to 60° C., treated with 4% seed crystals of crystalline R¹SO₂NHR² in 60:40 n-propanol:water, cooled to 0° C., and filtered. The filtrant was washed with 40:60 n-propanol:water (1.8 Kg/Kg product), dried at 45° C., and may be milled until amorphous.

Amorphous N-(2-((4-hydroxyphenyl)amino)pyridin-3-yl)-4-methoxybenzenesulfonamide is characterized by a plain halo in its powder diffraction pattern, an absence of peaks characteristic of a crystalline N-(2-((4-hydroxyphenyl)amino)pyridin-3-yl)-4-methoxybenzenesulfonamide, or both.

Therapeutic utility of N-(2-((4-hydroxyphenyl)amino)pyridin-3-yl)-4-methoxybenzenesulfonamide is demonstrated in commonly-owned U.S. application Ser. No. 10/857,235, May 28, 2004 and U.S. Application Ser. No. 60/575,577, May 28, 2004.

N-(2-((4-hydroxyphenyl)amino)pyridin-3-yl)-4-methoxybenzenesulfonamide binds to the colchicine site of tubulin β-subunits and inhibits the polymerization of tubulin. Accordingly, the compound is useful as a drug for treating diseases in a mammal which are caused or exacerbated by polymerization of tubulin. Such diseases include, but are not limited to, cancer and gouty arthritis, wherein cancer includes, but is not limited to, bone marrow dyscrasias, breast (ductal and lobular) cancer, cervical cancer, colon cancer, leukemia, lung (small cell and non-small cell) cancer, lymphoma, melonoma, mouth and tongue cancer, neuroblastoma (including pediatric neuroblastoma), pancreatic cancer, prostate cancer, rectal cancer, renal cancer, sarcoma, stomach cancer, uterine cancer, and cancers resulting from the metastasis of disease from these areas.

The term “mammal,” as used herein, means a particular class of vertebrate, preferably a human.

Compositions made with or comprising amorphous N-(2-((4-hydroxyphenyl)amino)pyridin-3-yl)-4-methoxybenzenesulfonamide may be administered, for example, bucally, ophthalmically, orally, osmotically, parenterally (intramuscularly, intraperintoneally, intrasternally, intravenously, subcutaneously), rectally, topically, transdermally, or vaginally. Ophthalmically administered dosage forms may be administered as, for example, elixirs, emulsions, microemulsions, oinments, solutions, suspensions, or syrups. Orally administered solid dosage forms may be administered as, for example, capsules, dragees, emulsions, granules, pills, powders, solutions, suspensions, tablets, microemulsions, elixirs, syrups, or powders for reconstitution. Osmotically and topically administered dosage forms may be administered as, for example, creams, gels, inhalants, lotions, ointments, pastes, or powders. Parenterally administered dosage forms may be administered, as, for example, aqueous or oleaginous solutions or suspensions. Rectally and vaginally dosage forms may be administered as, for example, creams, gels, lotions, ointments or pastes.

The therapeutically acceptable amount of amorphous N-(2-((4-hydroxyphenyl)amino)pyridin-3-yl)-4-methoxybenzenesulfonamide depends on recipient of treatment, the disease and severity thereof, the composition containing it, time of administration, route of administration, duration of treatment, its potency, its rate of clearance and whether or not another drug is co-administered. The amount of amorphous N-(2-((4-hydroxyphenyl)amino)pyridin-3-yl)-4-methoxybenzenesulfonamide used to make a composition to be administered daily to a patient in a single dose or in divided doses is from about 0.03 to about 200 mg/kg body weight. Single dose compositions contain these amounts or a combination of submultiples thereof.

Amorphous N-(2-((4-hydroxyphenyl)amino)pyridin-3-yl)-4-methoxybenzenesulfonamide may be administered with or without an excipient. Excipients include, but are not limited to, encapsulating materials and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof.

Excipients for preparation of compositions comprising amorphous N-(2-((4-hydroxyphenyl)amino)pyridin-3-yl)-4-methoxybenzenesulfonamide to be administered orally in solid dosage form include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof. Excipients for preparation of compositions made with amorphous N-(2-((4-hydroxyphenyl)amino)pyridin-3-yl)-4-methoxybenzenesulfonamide to be administered ophthalmically or orally in liquid dosage forms include, for example, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, and mixtures thereof. Excipients for preparation of compositions made with amorphous N-(2-((4-hydroxyphenyl)amino)pyridin-3-yl)-4-methoxybenzenesulfonamide to be administered osmotically include, for example, chlorofluorohydrocarbons, ethanol, water, and mixtures thereof. Excipients for preparation of compositions made with amorphous N-(2-((4-hydroxyphenyl)amino)pyridin-3-yl)-4-methoxybenzenesulfonamide to be administered parenterally include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for preparation of compositions made with or comprising amorphous N-(2-((4-hydroxyphenyl)amino)pyridin-3-yl)-4-methoxybenzenesulfonamide to be administered rectally or vaginally include, for example, cocoa butter, polyethylene glycol, wax, and mixtures thereof.

The foregoing is meant to be illustrative of the invention and not intended to limit it to the embodiments disclosed herein. Variations and changes obvious to one skilled in the art are intended to be within the scope and nature of the invention as defined in the claims.

What is claimed is:

1. Amorphous N-(2-((4-hydroxyphenyl)amino)pyridin-3-yl)-4-methoxybenzenesulfonamide.

2. A process for making substantially amorphous N-(2-((4-hydroxyphenyl)amino)pyridin-3-yl)-4-methoxybenzenesulfonamide, said process comprising milling N-(2-((4-hydroxyphenyl)amino)pyridin-3-yl)-4-methoxybenzenesulfonamide, with or without solvent, until the substantially amorphous N-(2-((4-hydroxyphenyl)amino)pyridin-3-yl)-4-methoxybenzenesulfonamide develops.

3. Amorphous R¹SO₂NHR², wherein R¹ is 4-methoxyphenyl and R² is (2Z)-((4-hydroxyphenyl)imino 1,2-dihydropyridin-3-yl prepared by the process of claim 2.
